(19) United States
(12) Patent Application Publication
(10) Pub. No.: US 2025/0198847 A1
(43) Pub. Date: Jun. 19, 2025

(54) OPTICAL MODULE

(71) Applicants: ams International AG, Jona (CH); THE JOHNS HOPKINS UNIVERSITY, Baltimore, MD (US)

(72) Inventors: Remco VERDOOLD, Geldrop (NL); Daniel NAJER, Rotkreuz (CH); Erik Jan LOUS, Veldhoven (NL); Piyush RAJ, Somerville, MA (US); Ishan BARMAN, Cockeysville, MD (US)

(21) Appl. No.: 18/867,026

(22) PCT Filed: Jan. 27, 2023
(86) PCT No.: PCT/EP2023/052041

§ 371 Date: Nov. 19, 2024

(30) Foreign Application Priority Data
May 20, 2022 (GB) 2207420.7

(51) Int. Cl. G01J 3/44; A61B 5/00; G01J 3/02; G01J 3/10; G01J 3/42; G01N 21/65

(52) U.S. Cl. CPC G01J 3/4412; A61B 5/0075; G01J 3/10; G01J 3/42; G01N 21/65; G01J 3/0227

(57) ABSTRACT

An optical module for spatially offset Raman spectroscopy, the optical module comprising: a laser source mounted on a substrate and configured to emit electromagnetic radiation at a target; a plurality of sensors mounted on the substrate and configured to detect electromagnetic radiation scattered from a plurality of depths in the target; and a first plurality of filters, each disposed over one or more of the plurality of sensors, wherein, the plurality of sensors and filters are arranged on the substrate at spatially offset positions from the laser source; and wherein the first plurality of filters are substantially transparent to a first wavelength band corresponding to a Raman scattering wavelength of a first molecule of the target and substantially opaque to wavelengths outside the first wavelength band.

### CROSS-REFERENCE TO RELATED APPLICATIONS

This is a national phase of International Application PCT/EP2023/052041, which was filed on Jan. 27, 2023, and which claims priority to British Application GB 2207420.7, which was filed on May 20, 2022, the entire contents of each of which are incorporated by reference.

### FIELD

The present disclosure relates to an optical module for spatially offset Raman spectroscopy and a method of use thereof.

### BACKGROUND

Raman spectroscopy is a technique that utilizes monochromatic electromagnetic energy produced by, for example, a laser to determine vibrational modes of molecules. A laser emits photons of a known and fixed wavelength that strike molecules bringing their energy levels to a virtual energy level state. After falling back from this state, the photon is scattered (emitted). When the energy levels of the laser emitted photon and the scattered photon correspond to each other (λ_{scatter}=λₗₐₛₑᵣ) the scattering is Rayleigh type scattering. Rayleigh scattering is the most observed type of scattering from an illuminated sample. A small fraction of the scattered photons will have a different photon energy and this type of scattering is Raman type scattering. In Raman scattering, a Stokes shift results in a higher wavelength number and anti-Stokes in a lower wavelength number.

If the illuminated sample contains a variety of different molecules, the observed Raman (Stokes and Anti-Stokes) scattering will have peaks spread over multiple wavenumbers and this may be visualized in a spectrum plot and analysed to identify the different molecules in the sample, each molecule being identifiable by the presence, position and strength of different peaks in the spectrum plot.

Known Raman spectrometers use a diffraction grating that divides the observed signal into multiple optical paths by wavelength, allowing the weaker Raman scattering signal to be isolated from the much stronger Rayleigh scattering signal. A line-pixel sensor may be connected to a lens to receive the Raman scattered light. A notch optical filter or a band pass filter may also be used to prevent the Rayleigh scattering component of the measured signal interfering with the much weaker Raman scattering component. A diffraction grating based optical setup such as this inevitably requires a minimum amount of space to ensure sufficient resolution is achieved when the diffraction grating divides the observed signal by wavelength. The greater the space requirements, the less a setup can be miniaturized.

One use of Raman spectroscopy is the analysis of biological and non-biological multi-layered samples as Raman spectroscopy allows Raman scattering signals from different layers to be isolated from each other, allowing readings of each of the layers to be made. For example, Raman spectroscopy may be used to measure hydration levels in different layers of a patient's skin by comparing the strength of Raman scattering signals from OH group molecules (associated with water) with those of CH₃ group molecules (associated with skin tissue) in each layer of the skin. A ratio of the strength of these two signals is indicative of a patient's hydration levels. Peter J. Caspers, Gerald W. Lucassen, Elizabeth A. Carter, Hajo A. Bruining, and Gerwin J. Puppels, (2001), “In Vivo Confocal Raman Microspectroscopy of the Skin: Noninvasive Determination of Molecular Concentration Profiles”, 2001, VOL. 116, NO. 3 Mar. 2001, IN VIVO RAMAN SPECTROSCOPY OF SKIN by “The Society for Investigative Dermatology, Inc.” (Caspers, 2001) proposes using a benchtop-sized diffraction grating based Raman spectrometer to determine water concentration in the dermis as a function of distance to the skin surface. In particular, at each probed depth under the skin surface, a ratio of the OH molecule concentration against the CH₃ molecule (present in biological dermis materials such as proteins) concentration may be determined by comparing the intensity of the Raman signal peaks (I_{water}) of OH molecules against the intensity (Iₚᵣₒₜₑᵢₙ) of CH₃-based molecules. The ratio gives an estimate of water content percentage at each probed depth and thus gives an indication of the patient's hydration levels.

In a similar manner by comparing ratios of Raman signals from different molecules, Raman spectroscopy may be used for determining the presence of other target analytes and relative concentrations or ratios thereof, for example, glucose, lactate, urea, cholesterol, keratin, collagen and so on.

The above methods however are not able to measure and correct for a patient's skin tone, type, thickness, and/or other physical variations such as, lipid content, hydration, hematocrit levels that result in heterogeneity associated the measured sample. Such heterogeneity results in interference in the measured Raman signals and may prevent an accurate and consistent measurement from being taken from different sample types. Further, correcting for these turbidity-induced distortions becomes particularly difficult because the associated parameters that govern the distortions have wavelength dependence. Therefore, different wavenumbers in the Raman spectra are exposed to different values of these parameters resulting in changes in intensity and distortions in line shape in the resultant spectra.

Spatially offset Raman spectroscopy (SORS) is a variant of Raman spectroscopy that allows for analysis of objects beneath obscuring surfaces. In a typical SORS measurement of a 2-layer system, at least two Raman measurements are taken. One at the source and one at an offset position. The two measured spectra can be subtracted using a scaled subtraction and the resulting spectra is representative of the subsurface scattering. For more complex systems, such as tissue, several spectra at varied offset distances may be measured and multi-variate analysis may be used to isolate measurements of the target layer(s) from the obscuring surface. As the spatial offset increases, the ratio of the spectral contribution from subsurface scattering increases at the cost of increased signal to noise ratio.

However, as with traditional Raman spectroscopy systems, SORS systems use a diffraction grating, benchtop sized optical fiber setups and/or other bulky optics to divide the scattered signal components according to their wavelength. Whilst this is useful if a full spectrum of molecular fingerprints is desired, it substantially increases space requirements limiting the scope for miniaturization

It is accordingly desired to be able to miniaturize a SORS system and account for target heterogeneity. These and other problems are solved by the present disclosure

### SUMMARY

In general terms, the inventors have realized that when the Raman spectra of only a smaller number of specific molecules in a target are analytes-of-interest, SORS may be performed using only filters corresponding to the Raman scattering wavelength(s) of those targets, without the need for bulky diffraction gratings and associated optical setups referred to above.

Specifically, SORS may be performed without a diffraction grating because only a narrow band or bands of the Raman shift spectrum is of interest, namely that or those associated with the Raman scattering of the specific set of analyte or analytes of interest in the target. These narrow bands can be isolated using suitable spectral filters without requiring a diffraction grating or complex optical fiber setups. This allows the entire SORS system to be miniaturized and provided in an integrated manner “on-chip”. Further, and synergistically, even a small reduction in the number of wavelengths analyzed when processing measured SORS spectra can substantially reduce the computational and hardware overhead required. Thus, selecting only a subset of wavelengths using a corresponding set of filters instead of from a full spectrum, allows the analysis of SORS spectra to be performed entirely “on-chip” by an integrated circuit, for example an ASIC without comprising analyte detection and measurement accuracy. Thus, the entire SORS system from the optics to the processing hardware used for signal analysis may be miniaturized. A miniaturized SORS system may accordingly be used in mobile and/or wearable devices such as a smart watch or smart phone.

Example specific uses of the present disclosure accordingly include determining relative concentrations of two or more target molecules, for example: ratios between CH3 and OH molecules to measure hydration in a patient's skin, and ratios between collagen and keratin at different depths of a patient's skin to determine thickness of the dermis and epidermis layers, as well as identifying concentrations of other analytes such as glucose, urea and so on.

Thus, according to a first aspect, there is provided an optical module (200) for spatially offset Raman spectroscopy, the optical module (200) comprising: a laser source (201) mounted on a substrate (202) and configured to emit electromagnetic radiation (203) at a target (204); a plurality of sensors (206) mounted on the substrate (202) and configured to detect electromagnetic radiation (207) scattered from a plurality of depths in the target (204); and a first plurality of filters (208), each disposed over one or more of the plurality of sensors (206), wherein, the plurality of sensors (207) and filters (208) are arranged on the substrate at spatially offset positions from the laser source (201); and wherein the first plurality of filters (208) are substantially transparent to a first wavelength band corresponding to a Raman scattering wavelength of a first molecule of the target (204) and substantially opaque to wavelengths outside the first wavelength band.

Optionally, the substrate of the optical module comprises an integrated circuit configured to:
determine a photon count at each of the plurality of sensors from the detected electromagnetic radiation at each of the plurality of sensors; and use the determined photon counts to estimate an absorption coefficient and a reduced scattering coefficient of the target (204) at each spatially offset position.

Advantageously, the inventors have realized that absorption and reduced scattering coefficients of the target may be efficiently and effectively estimated using only photon count information from each sensor without needing to make other measurements and without other a priori information about the target being measured. As will be appreciated, absorption and reduced scattering coefficients provide a fingerprint of scattering behavior of the target. Accordingly, if these coefficients can be estimated accurately and for each wavelength (as determined by the chosen spectral filters), a comparison can be made against samples with similar absorption and reduced scattering coefficients in training data for which physical information (such as physical composition, optical parameters and dimensions) is known and such information about the target may then be inferred. Through this approach, the heterogeneity of the target is implicitly accounted for in the photon counts of both the training data and the target data and thus in the estimated absorption coefficient and reduced scattering coefficients.

Specifically, as described above, in a two or more-layer system where the ‘to be detected’ specimens are heterogeneous, there is no straightforward approach to simultaneously measuring the absorption coefficient and the reduced scattering coefficient of the different layers from a single Raman spectrum. This is typically because the target's turbidity (i.e. interplay of absorption and scattering) leads to non-analyte specific variations in the Raman measurement and specifically to intensity and line-shape distortions. This diminishes analyte concentration prediction accuracy and the accuracy of estimations of other physical parameters of the target.

However, photon count values at each offset position are implicitly affected by heterogeneity of the target (as photon counts at each offset position depend on how much scattering has occurred from each layer in the target, as well as how much of the emitted laser source light reaches the sensor at that position). Accordingly, absorption and reduced scattering coefficients estimated using measured photon counts also implicitly account for this heterogeneity. The inventors have realized a model may be accordingly be trained to make this estimation using only photon count data in which the heterogeneity is implicitly also taken into account. Thus providing a way to accurately estimate absorption and reduced scattering coefficients while taking into account the heterogeneity of the target.

In a non-limiting example, a non-linear regression model, based on one or more machine learning approaches including, but not limited to, support vector machines and deep learning, may be built with inputs from known sample measurements as training data. The training data may accordingly comprise data from the known samples of known absorption and reduced scattering coefficients in association with photon counts at different spatial offsets, physical parameters (e.g. in the case of a skin sample, dermis and epidermis thicknesses and skin tone), composition parameters (e.g. in the case of a skin sample, collagen concentration, keratin concentration), and any other data representative of the type of target on which the model is to be used.

Thus, optionally the integrated circuit is configured to apply a trained model to said determined photon counts to estimate said absorption coefficient and reduced scattering coefficient of the target (204) at each spatially offset position.

For example, in the case of a skin tissue target of unknown dermis/epidermis layer thicknesses and skin tone, absorption and reduced scattering coefficients prediction error rates were achieved as small as 7-13% where the training data contained a sufficiently large and representative sample of photon counts associated with different target types. This accuracy was further improved to an error rate of around 1-5% by including scattering measurements at multiple illumination wavelengths at each of the offset positions in the measurement and training data.

Further, the process of measuring photon counts and applying a trained model stored onto a memory of the integrated circuit to the measured photon counts is computationally simple compared to known SORS analysis methods so synergistically allows the absorption and reduced scattering coefficients predictions to be entirely performed “on-chip” without the substantial computational resources and hardware requirements of known SORS systems. This enables miniaturization of the entire system and not only the optics.

Optionally, the integrated circuit is further configured to: compare the estimated absorption coefficients and/or reduced scattering coefficients of the target (204) with previously determined absorption coefficients and/or reduced scattering coefficients associated with one or more known samples to infer physical information about the target (204).

As described above, absorption and reduced scattering coefficients are an indicative fingerprint of the scattering behavior of the target so known samples with similar absorption and reduced scattering coefficients are likely to have similar physical parameters. Accordingly, physical information about the target may be inferred by comparing the estimated absorption and reduced scattering coefficients with absorption and reduced scattering coefficients of known samples.

Where these are similar, the target is likely to have similar physical properties. For example, in the skin tissue example, measured absorption and reduced scattering coefficients may be similar to those of a known sample of a light skin type with a known dermis and epidermis thickness. It can thereby be inferred that the target is likely to be of a similar skin type and have similar dermis and epidermis thicknesses.

Optionally, the integrated circuit is further configured to: identify which of the plurality of sensors (206) has a highest signal to noise ratio for the detected Raman scattering signals associated with said first molecule.

For example, in the case of skin tissue, it is desired that information from the dermis is collected and not from the epidermis and/or pigment layer. In the skin system the latter two can be considered as a single layer. Information from this effective layer needs somehow to be removed from the measurement to isolate information about the dermis. As described above, in SORS, the larger the spatial offset, the deeper in the dermis the information is collected from. However, the question remains: at which spatial offset best spectrum be obtained where the signal from the dermis layer is strongest? The answer will vary between different skin tissue targets due to heterogeneity.

However, it is known that the epidermis is predominantly populated by keratin while dermis has an abundance of collagen. The spectra for collagen is stronger in the wavenumber region (800-1000 cm⁻¹) compared to keratin. Accordingly, the offset distance where the keratin Raman signal level falls below that of the collagen level indicates that that is where the epidermis layer ends and the dermis layer starts. Thus, the spectra may be analysed for this inversion of the keratin signal to collagen signal ratio. The depth corresponding to the offset distance where this occurs is indicative of the thickness of the epidermis. Thus, where only the dermis layer is of interest and any data from the epidermis is to be disregarded, the spectra measured by the sensors at offsets where the keratin signal level is higher than the collagen level can be disregarded from any subsequent analysis of the target.

It is envisaged that, once the sensor(s) with the strongest signal to noise ratio have been identified, known SORS weighted subtraction and/or multi-variate analysis techniques may be applied by the integrated circuit for each measured wavelength to isolate the subsurface scattering signal(s) from the surface signal as will be appreciated by the skilled person. For example, known algorithms such as MCR (Multivariate Curve Resolution) and BTEM (Band-target entropy minimization) are envisaged to find individual component spectra in multi-component sample measurements to establish the spectral contributions of each layer and, hence, quantify the compositional contributors for that. Specifically, MCR achieves better results with spectra measured at fixed locations and BTEM achieves better results with spectra taken at multiple offset locations. Other envisaged methods to isolate individual component spectra include pprincipal component analysis and non-linear machine learning and deep learning approaches.

Optionally, the integrated circuit is further configured to estimate a turbidity of the target (204) and correct an output signal of one or more of said plurality of sensors (206) to compensate for said estimated turbidity.

As described above, absorption and reduced scattering coefficients are an indicative fingerprint of the scattering behavior of the target and together define the turbidity of the target. Thus, once the absorption and reduced scattering coefficients are estimated, corrections to intensity and distortions in line shape in the resultant measured spectra may be estimated and applied to one or more of the measured signals. For example, is envisaged that one or more of the methods described in “Turbidity-Corrected Raman Spectroscopy for Blood Analyte Detection, I. Barman et al, Analytical Chemistry, Vol. 81, No. 11, 1 Jun. 2009” may be used to estimate and correct for a turbidity Optionally, the integrated circuit is further configured to identify a modulating component in an output signal of one or more of said plurality of sensors associated with a periodic change in dimensions and/or composition of the target (204).

As will be appreciated, some targets may experience periodic change in dimensions and/or composition. For example, if the target is skin tissue, blood supply pulses to and from veins in the skin. This can have an effect on physical dimensions (i.e. expansion and contraction of one or more layers as blood flows into and out of the sample), as well as changes in composition (i.e. circulating blood may bring higher concentrations of analyte into/out from the sample). These changes, also described as tissue modulation, typically occur periodically and thus may be identified in the measured spectra as mobile components (as opposed to immobile components which do not undergo such changes). It is envisaged that one or more of the methods described in “Utilizing pulse dynamics for non-invasive Raman spectroscopy of blood analytes, M. S. Wrobel et al, Biosensors and Bioelectronics, Volume 180, 15 May 2021, 113115” may be used to identify one or more of said modulating components in the measured signals.

In particular, it is envisaged that the natural pulse rhythm may be derived with the measured Raman data and a phase-sensitive detection algorithm applied to enhance the signal of the mobile components. In particular, it is envisaged that method may comprise detection of the pulse frequency and performing a filtering of the periodical signal with a digital phase-detection algorithm, resulting in the estimation of the pulse-correlated Raman signal. In the case where the target is skin tissue, this method is particularly advantageous as it is largely independent of other target-specific variations because optical parameters and chemical composition of blood are much more consistent over the human population of different races and ages than that of the surrounding solid tissue.

Optionally, said laser source (201), said plurality of sensors (206), and said plurality of filters (208) are integrated with said integrated circuit, and/or the integrated circuit may comprise comprises an ASIC.

Advantageously, as described above, integration of all of the SORS system components into an integrated circuit onto a single substrate allows SORS measurements and analysis to be performed entirely “on-chip”.

Optionally, the plurality of sensors (206) are arranged in a line beginning at the laser source (201). Additionally or alternatively, the plurality of sensors (206) may be arranged in a plurality of radial directions around the laser source (201) in concentric, center symmetric patterns.

As described above, SORS requires measurements at a plurality of offsets from the laser source. The sensors may accordingly be arranged in a single line from the laser source or, to advantageously increase coverage and thus signal capture, in multiple lines in different directions emanating from the laser source thereby defining one or more concentric, center symmetric patterns. This arrangement also allows different quadrants or other divisions of the pattern to be covered with different spectral filters, each quadrant or other division thus providing the option of a different wavelength measurement but having the same set of offset distances from the laser source. In other words, the plurality of sensors in each radial direction may have the same wavelength filter to capture one wavelength/wavenumber in the Raman spectrum.

Optionally, the plurality of sensors (206) are arranged at equal distances from each other in said line or in each said radial direction. Alternatively, the plurality of sensors (206) may be arranged at unequal distances from each other in said line or in each said radial direction.

For example, the offsets may correspond to 1-multiples of wavelengths of one or more of the Raman spectrum wavelengths that are intended to be measured which may be in the range of e.g. around 0.1-2 mm.

Optionally, the plurality of sensors comprises at least three sensors in said line or in each said radial direction.

Advantageously, the greater the number of sensors, the greater the resolution of the measurements. Accordingly, it is also envisaged that there may be at least five, ten, fifteen, twenty or more sensors.

Optionally, the optical module comprises further pluralities of filters, each disposed over one or more of the plurality of sensors (206), wherein the further pluralities of filters are substantially transparent to a further wavelength bands corresponding to a Raman scattering wavelength of further molecules of the target and substantially opaque to wavelengths outside said further wavelength bands.

As described above, whilst it is not envisaged that full, continuous Raman spectra are captured, it is envisaged that the SORS spectra of at least a multiple Raman wavelengths/wavenumbers are captured, according to the specific application. For example, if hydration is to be measured, at least two pluralities of filters are used (one set corresponding to the Raman scattering wavelength of OH molecules and one set corresponding to the Raman scattering wavelength of CH3 molecules) so that measurements at each of the offset positions in each of the wavelengths may be made. Accordingly, each of the further plurality of filters corresponds to a Raman scattering wavelength of a desired target molecule. Other examples will require suitable numbers of other spectral filters.

Optionally, the integrated circuit is configured to: estimate a ratio of signal strength between a detected Raman scattering signal of the first molecule and of one or more of said further molecules of the target (204) at each of said spatially offset positions; and compare the estimated ratios with previously determined ratios associated with one or more samples of known physical parameters to estimate one or more physical parameters of the target (204). The physical parameters may be, for example, a thickness of one or more layers of the target (204).

Advantageously, the measured signals may be used to determine relative concentrations of multiple molecules in the target and use these to estimate further information about the target. By way of illustrative example, a first plurality of filters may be transparent to a wavelength band corresponding to a Raman scattering wavelength of an OH molecule and a second plurality of filters is transparent to a wavelength band corresponding to a Raman scattering wavelength of a CH₃ molecule. The ratio of the peaks between these two signals is indicative of hydration levels in the target. The SORS spectra (i.e. at all offset distances) may be obtained as described above. The offset (i.e. sensor) with the strongest signal to noise ratio (i.e. that with the highest peak values) may be selected as described above. Further, if multiple laser output wavelengths are used, for example wavelengths between 600-785 nm or between 500-900n, the spectra obtained from the wavelength with the strongest signal to noise ratio may be selected. Once the optimum spectra is determined, the ratio of the signal strength between the two molecules is estimated to provide an indication of hydration levels. By using the methods of the present disclosure hydration may accordingly be determined in a manner that is independent of target heterogeneity. A further illustrative example is calculating the ratio of collagen to keratin signals as described above to determine a thickness of the dermis and/or epidermis layers of a skin tissue target. Other analyte concentrations and ratios are also envisaged.

Thus, advantageously, as described above, the present disclosure allows physical parameters of a target to be determined cheaply and with a miniaturized device and independently of target heterogeneity.

Optionally, the laser source (201) is configured to emit electromagnetic radiation in a plurality of wavelengths, and wherein the integrated circuit is further configured to: estimate said absorption coefficient and a reduced scattering coefficient of the target (204) from determined photon counts for each of said plurality of wavelengths.

As described above, if there are multiple analytes, it may be necessary to provide broadband illumination by the laser source or laser sources to ensure the target analytes are sufficiently illuminated at an appropriate wavelength. Further, as described above, this may result in an increase in accuracy of the absorption and reduced scattering coefficients, reducing the error rate to at least 1-5%.

Optionally, the first plurality of filters (208) are disposed over one or more of the plurality of sensors (206) in a first radial direction from the laser source (201), and wherein the further plurality of filters are disposed over one or more of the plurality of sensors (206) in further radial directions from the laser source (201).

As described above, this arrangement advantageously allows different angular segments around the laser source to be used to measure different wavelengths at the same set of offset positions.

Optionally, the optical module comprises one or more lenses (210), each disposed over a respective one of the laser source (201) and/or plurality of sensors (206).

Advantageously, the lens increases signal to noise ratio by focusing scattered light directly onto the plurality of sensors and focusing the laser light from the laser source onto a suitable depth on or in the target.

Optionally, said first and/or further plurality of filters (208) comprise angular filters defining a field of view for each sensor of less than 28 degrees.

Advantageously, the angular filter allows rays at angle of less than 14° from the optical axis to pass through to the underlying sensor, thereby providing a field of view of 28° for each sensor. The inventors have found that this field of view maximizes signal to noise at each sensor.

Optionally, the integrated circuit (202a) is further configured to: control the laser source (201) to emit modulated electromagnetic radiation at the target (204) and to demodulate the detected electromagnetic radiation scattered from the target (204).

Advantageously, as Raman scattering is a fast process, any modulation of, for example the frequency, amplitude, and/or phase of the laser source may also be detected in the scattered signal incident on the plurality of sensors. This allows for phase-locked detection of the modulation frequency in the Raman scattering signal, which further improves the sensitivity and accuracy of the detection of the Raman scattering signal.

For example, sensitivity may be increased by 2-3 orders of magnitude independently of background environment lighting conditions (such as daylight). Accordingly, this allows the optical module to perform Raman spectroscopy in settings where lighting conditions cannot be easily be controlled such as point of care and sporting environments.

In one implementation, the integrated circuit accordingly comprises phase lock loop, amplification and laser source driver circuitry to provide the above increase in sensitivity. Advantageously, providing the phase lock loop, amplification and laser source driver circuitry on a single integrated circuit together with the plurality of sensors and filters also simplifies the electronic and optical design thereby reducing parasitic effects and noise compared to more complex circuits.

According to a second aspect of the present disclosure, there is provided a method of performing SORS on a target using the optical module (200) of any preceding claim.

The method provides the same advantages as the first aspect of the disclosure described herein.

### BRIEF DESCRIPTION OF THE DRAWINGS

These and other aspects of the present disclosure will now be further described, by way of example only, with reference to the accompanying figures in which:

FIG. 1 shows a known plot of known Raman spectra.

FIG. 2a illustratively shows an optical module according to the present disclosure.

FIG. 2b illustratively shows an optical module according to the present disclosure.

FIG. 3a illustratively shows an optical module according to the present disclosure.

FIG. 3b illustratively shows an optical module according to the present disclosure.

FIG. 3c illustratively shows an optical module according to the present disclosure.

FIG. 3d illustratively shows an optical module according to the present disclosure.

FIG. 4 illustratively shows a portion of an optical module according to the present disclosure.

FIG. 5a shows that the number of scattered photons at the sensors/collectors positioned further from the laser source/emitter is less than the number of photons at the sensors/collectors positioned closest to the laser source/emitter.

FIG. 5b shows a top view of the simulation described in.

FIG. 6a is a graph showing the fraction of light collected against offset depth in the target for the simulation of FIGS. 5a and 5b.

FIG. 6b is a graph showing percentage of photons emitted by the laser source which are collected as scattering by the sensors against spatial offset.

FIG. 7 is a graph showing normalised photon intensity against scattering coefficient.

FIG. 8a is a graph showing predicted absorption coefficient against true absorption coefficient of a one-layer homogeneous target model.

FIG. 8b is a graph showing predicted reduced scattering coefficient against true reduced scattering coefficient of a one-layer homogeneous target model.

FIG. 9a is a graph showing predicted absorption coefficient against true absorption coefficient of a two-layer target model.

FIG. 9b is a graph showing predicted reduced scattering coefficient against true reduced scattering coefficient of a two-layer target model.

FIG. 10a is a graph showing predicted absorption coefficient against true absorption coefficient of a two-layer target model.

FIG. 10b is a graph showing predicted reduced scattering coefficient against true reduced scattering coefficient of a two-layer target model.

FIG. 11a is a graph showing predicted absorption coefficient against true absorption coefficient of a two-layer target model.

FIG. 11b is a graph showing predicted reduced scattering coefficient against true reduced scattering coefficient of a two-layer target model.

FIG. 12a is a graph showing predicted absorption coefficient against true absorption coefficient of a two-layer target model.

FIG. 12b is a graph showing predicted reduced scattering coefficient against true reduced scattering coefficient of a two-layer target model.

Like elements are indicated by like reference numerals.

### DETAILED DESCRIPTION

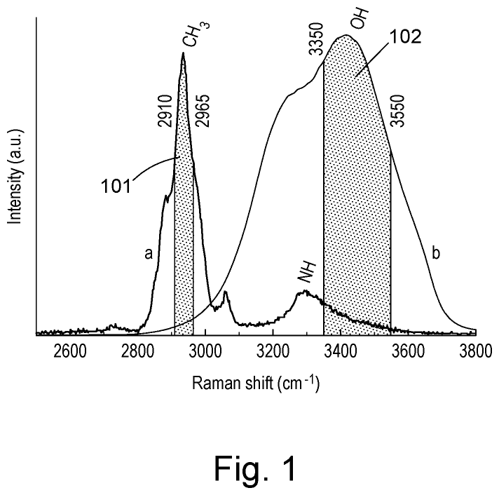

FIG. 1 indicates known Raman spectra of a probed portion of a target, for example human skin. There are peaks 101, 102 in the spectra at, for example, the Raman shifts associated with CH3, OH and NH molecules that make up a large proportion of human skin tissue. As described above, where it is already known what analyte is being investigated in a target, it is not necessary to obtain full spectra as shown in FIG. 1. Instead, spectral filters may be used to isolate the wavelengths associated with Raman shifts of the already identified analytes, thereby enabling the miniaturization described above.

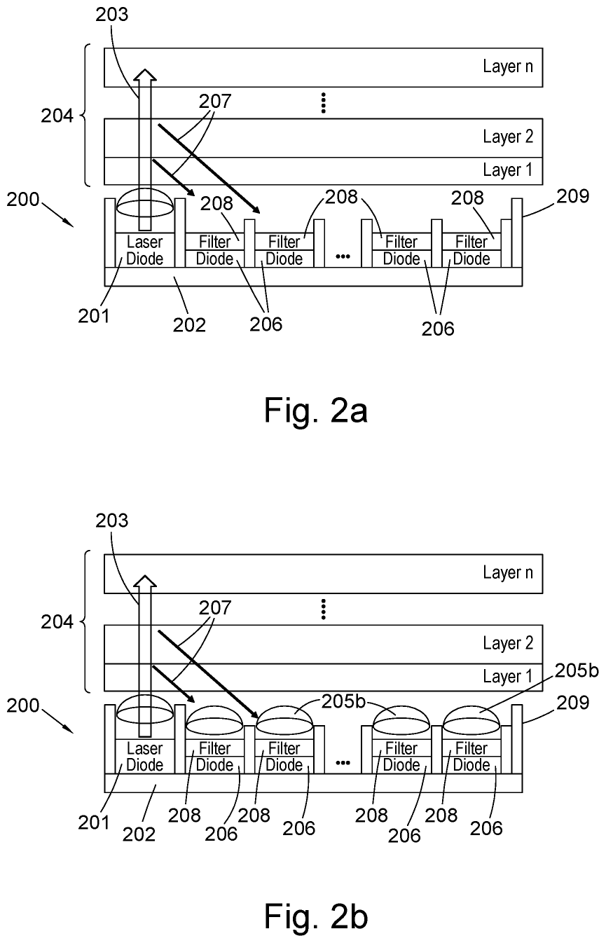

FIG. 2a shows an optical module according to the present disclosure. The optical module comprises a laser source 201 mounted on a substrate 202. The laser source is configured to emit electromagnetic radiation 203 at a target 204 that may comprise any number n of different layers.

The laser source 201 may be, for example, a laser diode or a VCSEL. The optical module further comprises a plurality of collector sensors 206 mounted on the substrate 202 and configured to detect electromagnetic radiation scattered from a plurality of depths (i.e. from the different n layers) in the target 204. The sensors in FIG. 1 are photo diodes but it is envisaged that any other suitable sensor type may also be used. The optical module further comprises a first plurality of filters 208, each disposed over one or more of the plurality of sensors 206. An optional lens 205a positioned over the laser source is also provided to focus the electromagnetic energy emitted by the laser source 201 onto a suitable plane or planes in the target 204.

The plurality of sensors 206 and filters 208 arranged thereover are positioned on the substrate 202 at spatially offset positions from the laser source 201. The greater the offset from the laser source 201, the greater the contribution the scattering from deeper layers has to the measured signal. The dots in FIG. 2a indicate that any number of sensors with filters thereover are envisaged and any number of layers in the target are envisaged.

The first plurality of filters 208 are substantially transparent to a first wavelength band corresponding to a Raman scattering wavelength of a first molecule of the target 204 and substantially opaque to wavelengths outside the first wavelength band. In this way, the Raman scattering signals from the desired target analyte may be isolated both from Raman scattering signals of other molecules and from noise of any other wavelength.

The optical module 200 is further provided with support structures 209 positioned on and around the laser source 201, sensors 206 and filters 208. The support structures may define apertures through which electromagnetic radiation 203 from the laser source 201 reaches the target 204 and through which scattered electromagnetic radiation 207 reaches the sensors 206.

FIG. 2b illustratively shows an optical module 200 according to the present disclosure. Like-numbered reference numerals refer to like elements. The optical module 200 in FIG. 2b is the same as in FIG. 2a but is also provided with optional lenses 205b positioned over each of the sensors and filters to focus electromagnetic radiation 207 scattered from the layers of the target 204 onto the sensors through the filters. The support structures 209 in also secure the lenses 205a, 205b in their respective positions.

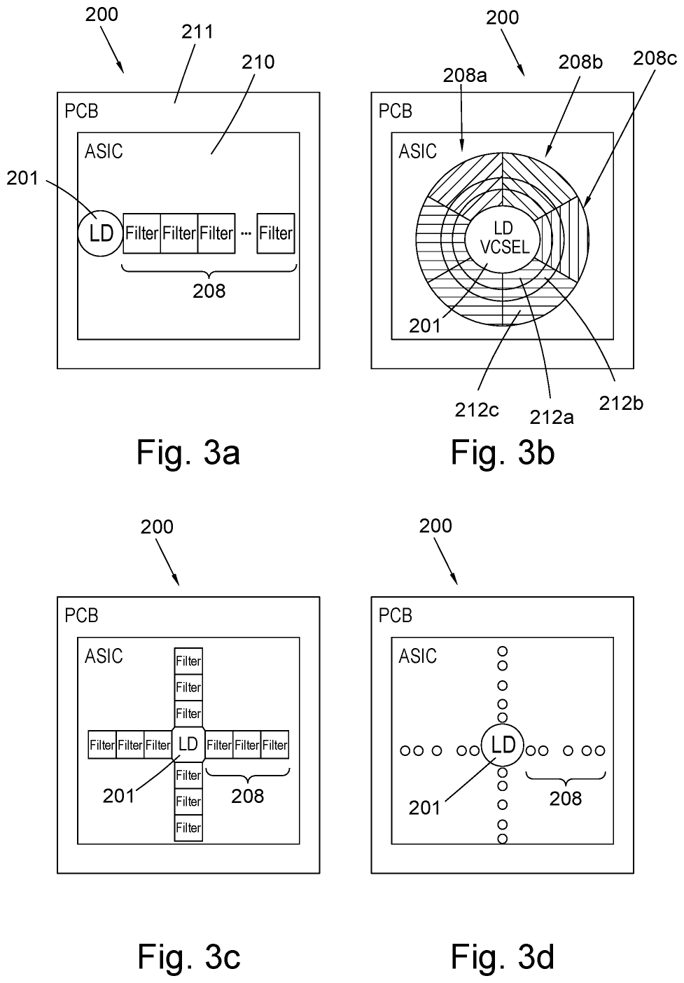

FIGS. 3a, 3b, 3c, 3d illustratively show top views of optical modules 200 according to the present disclosure. Like-numbered reference numerals refer to like elements.

The optical module 200 in FIG. 3a has a layout corresponding to that shown in FIGS. 2a and 2b whereby a laser source 201 is mounted on a substrate, in this case an ASIC 210 on a printed circuit board (PCB) 211. The plurality of sensors positioned under the filters 208 are not visible but are rectangular collector diodes positioned immediately adjacent each other.

FIGS. 3b, 3c, 3d show the optical module 200 but with different layouts of filters and sensors.

In FIG. 3b, the sensors are arranged around the laser source 201 which is positioned centrally on the substrate. The sensors define center-symmetric concentric rings 212a, 212b, 212c around the laser source 201 with correspondingly shaped filters positioned thereover. The filters may further be divided into sets 208a, 208b, 208c corresponding to different quadrants or other radial divisions around the laser source 201. The collector diodes may accordingly have a corresponding partial annulus shape or design to match the shape of the concentric rings. Each set corresponding to a different. In this way, multiple spatially offset Raman spectra of different wavelengths may be obtained from data collected at the same set of sensor offsets. The number of different filters sets will corresponds to the number of target analytes being investigated. In the example of FIG. 3b, there are six radial divisions providing for the measurement of six different wavelengths. Other numbers of radial divisions are also envisaged.

In FIG. 3c, the layout is similar to that of FIG. 3a but additional perpendicular lines of sensors 208 and filters are also provided in additional directions relative to the laser source 201. This layout provides similar functionality to that of FIG. 3b in that multiple Raman spectra may be obtained at different wavelengths through the sensor sets in each of the different lines. The filters may accordingly have different spectral ranges. In FIG. 3c, the sensors are equally spaced apart form each other. The sensors in FIG. 3c have a rectangular design.

In FIG. 3d, the layout is the same as that of FIG. 3c, except that the sensors are spaced at unequal distances from each other, for example, at distances matching 1-multiple wavelengths of the electromagnetic radiation emitted by the laser source 201. The sensors in FIG. 3d have a circular design but a rectangular design is also envisaged.

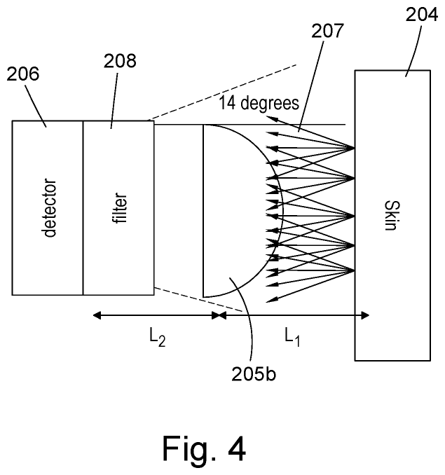

FIG. 4 illustratively shows a portion of an optical module according to the present disclosure, namely one of the plurality of sensors 206 and filters 208 with a lens 205b positioned thereover and positioned a distance L1+L2 away from the target 204, for example skin tissue. Like elements are indicated by like reference numerals. The filter 208 in FIG. 4 is provided as an angular filter configured to allow only electromagnetic radiation 207 scattered from the target 204 through to the sensor 206 if an angle of incidence is 14 degrees or less from the optical axis thereby providing a field of view of 28 degrees. In order to maximise signal to noise ratio, the distance L2 between the lens 205b and the sensor 206 may be varied during manufacture whereas it is envisaged that the distance L1 between the lens 205b and the target 204 is fixed and is instead determined by how close the optical module is to the target 204. For example, it is envisaged that placement of the optical module may be as close as around 200 μm to avoid pinching effects during measurements. By selecting a lens 205b with a suitable radius of curvature, setting the appropriate dimensions of the optical module in this manner and by providing angular selectivity through the filter, increases in detected signal of ˜34% are achieved compared to the optical module without any lens positioned over the filter 206. Given that Raman scattering signal strength is already low relative to other scattering signals, and given that increased spatial offsets of the sensors further reduces this, an increase in 34% in photons collected is particularly advantageous.

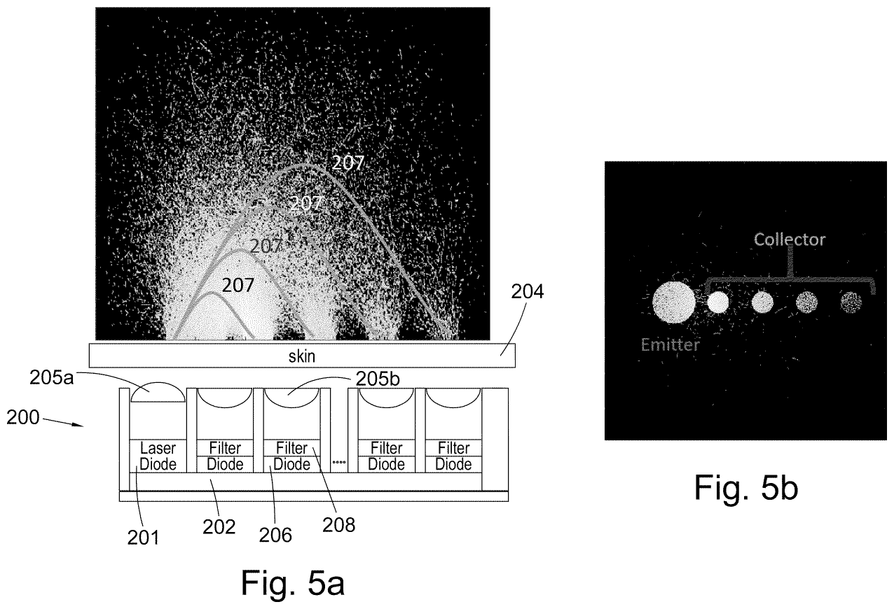

FIG. 5 illustratively shows an optical module according to the present disclosure during simulated operation, for example, the optical module of FIG. 2b. Like elements are indicated by like reference numerals. As is shown in FIG. 5, electromagnetic radiation 207 from the laser source 201, in this example a VCSEL, is scattered from different layers in the target. The detected signals at the different sensors 206 at each the different offsets will be made up of contributions from different layers of the target 204. The greater the offset from the laser source 201, the greater the contribution from deeper layers and vice versa.

FIG. 5b illustratively shows a top view of the simulation described in FIG. 5a and illustrates that the number of scattered photons at the sensors/collectors positioned further from the laser source/emitter is less than the number of photons at the sensors/collectors positioned closest to the laser source/emitter.

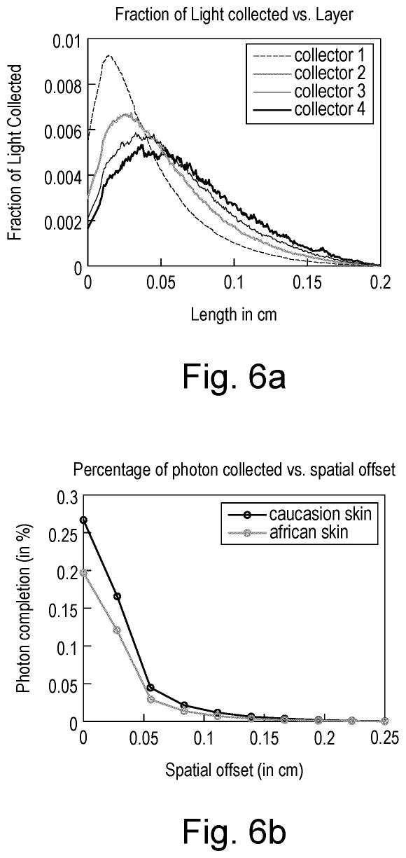

FIG. 6a is a graph showing the fraction of light collected against offset depth in the target for the simulation of FIGS. 5a and 5b. The graph shows that the fraction of light from shallow depths contributes most to the signal of the sensor (collector 1) closest to the laser source whereas each successive sensor (collectors, 2, 3 4) has an increasing fraction of collected light arising from deeper layers in the target.

FIG. 6b is a graph showing percentage of photons emitted by the laser source which are collected as scattering by the sensors against spatial offset of those sensors for two different target types, namely Caucasian skin tissue and African skin tissue. The graph in FIG. 6b shows that fewer photons are collected when the target is African skin tissue. This is because the absorption coefficient is higher in African skin tissue. Accordingly, from this graph, it is apparent that the photon counts at the sensors provide an indication as to the physical parameters of the target.

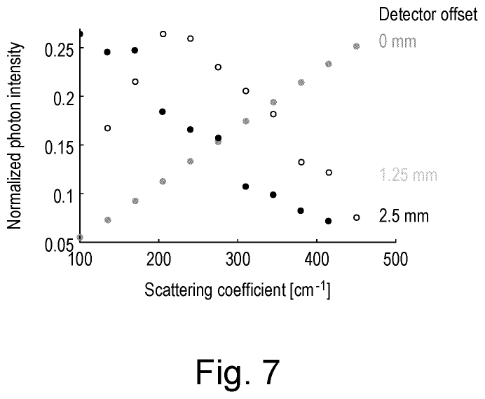

FIG. 7 is a graph showing normalised photon intensity against scattering coefficient for three different detector offsets of a simulation. The offsets are 0 mm (i.e. at the position of the laser source), 1.25 mm and 2.5 mm. From this graph it is apparent that as the scattering increases, the signal at the sensors increases. It is also apparent that as the spatial offset increases, increased scattering causes reduction of total photon collected because more of the photons are lost (absorbed or scattered away from the sensors) in deeper layers.

It will be appreciated that, if there is no scattering (i.e. a zero scattering coefficient), all the photons emitted by the laser source will travel uninterrupted in the direction of propagation. Some of them might get absorbed, but almost none of them will scatter back to the sensors. This kind of situation usually arise in perfectly clear targets, for example clear liquid solutions. As scattering (i.e. scattering coefficient) increases, more and more photons are detected at the sensors. Beyond a certain scattering coefficient value, the signal at the collectors saturates, for example, because the geometry of the sensors limits the total light that can enter the sensors.

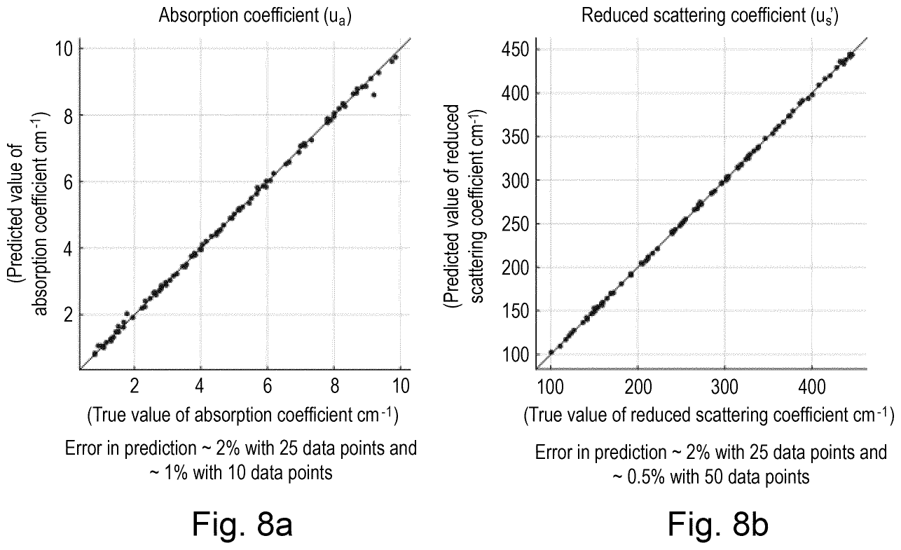

Because of these two trends (i.e. (i) increased absorption reducing photon counts, and (ii) increased scattering increasing photon counts up to a saturation value) the percentage of photons collected at different spatial offset (i.e. the photon count) can be used to predict absorption and reduced scattering coefficients of a target with a high level of accuracy. This is achieved by training a model in the manner as described above to predict absorption and reduced scattering coefficients using only photon count datasets. The effectiveness of this approach is demonstrated in the graphs shown in FIGS. 8a-12b.

FIG. 8a is a graph showing predicted absorption coefficient against true absorption coefficient of a one-layer homogeneous target model and FIG. 8b is a graph showing predicted reduced scattering coefficient against true reduced scattering coefficient of the model. The data from which the graphs are generated is collected at sensor offsets of 0 μm, 250 μm, 500 μm, 750 μm, 1 mm, 1.25 mm, 1.5 mm, 1.5 mm, 1.75 mm, 2 mm, 2.25 mm, 2.5 mm. In this setup, a laser with Gaussian distribution illuminates the target and the number of photons collected at the sensors at the different offsets is stored. These measurements are performed for varying values of absorption (uₐ) and reduced scattering coefficient (uₛ′) (both in cm⁻¹) to collect a training data set. Once training dataset of spatial offset location with number of photons arriving at the collector is sufficiently large, the model is trained to predict absorption and reduced scattering coefficient using the training data set, i.e. using only the photon count dataset. In the example graph of FIGS. 8a and 8b, a 5-fold cross validation approach is used.

As expected, as the target of FIGS. 8a and 8b is a homogenous target with a single layer, the predicted absorption and reduced scattering coefficients are very close and the error rate is roughly 1-2% depending on the number of data points used.

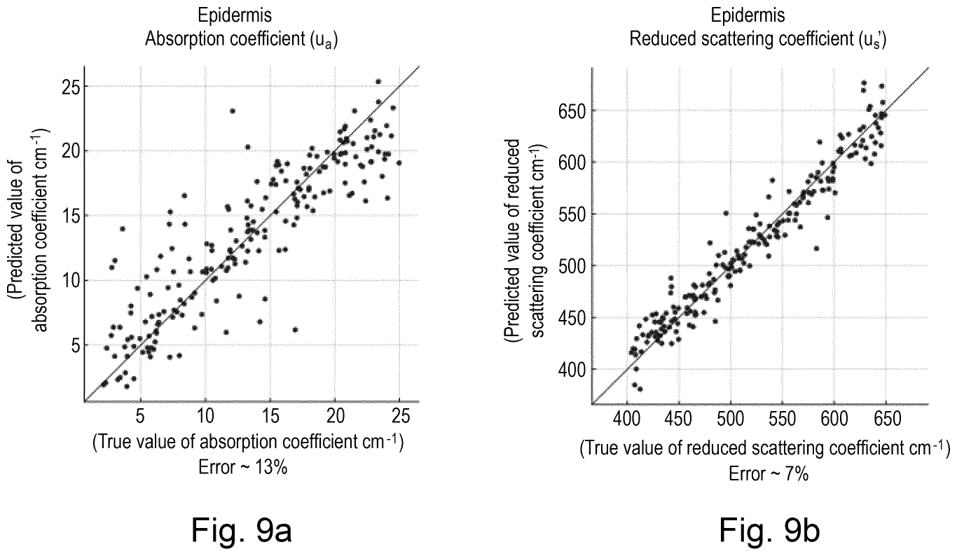

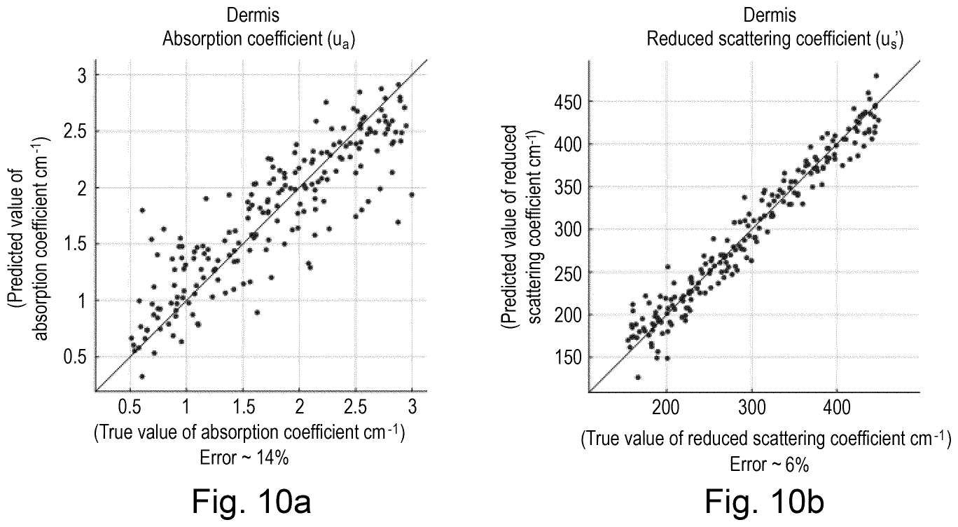

FIGS. 9a, 9b, 10a, 10b respectively show similar graphs but now the target is a two-layer model, each layer having fixed thicknesses. For example, the example target used is a target having a fixed thickness dermis and fixed thickness epidermis. Each layer has its own absorption coefficient and its own reduced scattering coefficient which must be predicted. As before, the data from which the graphs are generated is collected at sensor offsets of 0 μm, 250 μm, 500 μm, 750 μm, 1 mm, 1.25 mm, 1.5 mm, 1.5 mm, 1.75 mm, 2 mm, 2.25 mm, 2.5 mm. A laser with Gaussian distribution illuminates the target and the number of photons collected at the sensors at the different offsets is stored. These measurements are performed for varying values of absorption (uₐ) and reduced scattering coefficient (uₛ′) (both in cm⁻¹) to collect a training data set. Once training dataset of spatial offset location with number of photons arriving at the collector is sufficiently large, the model is trained to predict absorption and reduced scattering coefficient using the training data set, i.e. using only the photon count dataset. In the example graphs of FIGS. 9a-10b, a 5-fold cross validation approach is used.

It can be seen from the graphs in FIGS. 9a-10b that the error rate has increased for both absorption coefficient and reduced scattering coefficient predictions of both the dermis and epidermis layers compared to the ideal one-layer model. The error rates are respectively 13%, 7%, 14% and 6%. However, these error rates are still low enough allow a good approximation of the coefficient values to be made. The advantage accordingly still remains that the predictions are made using only photon counts, which are easily measured using the optical module of the present disclosure.

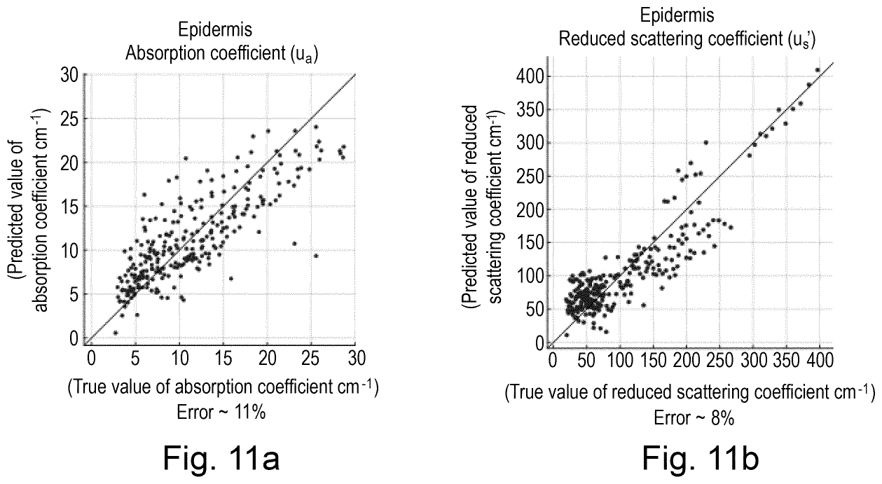

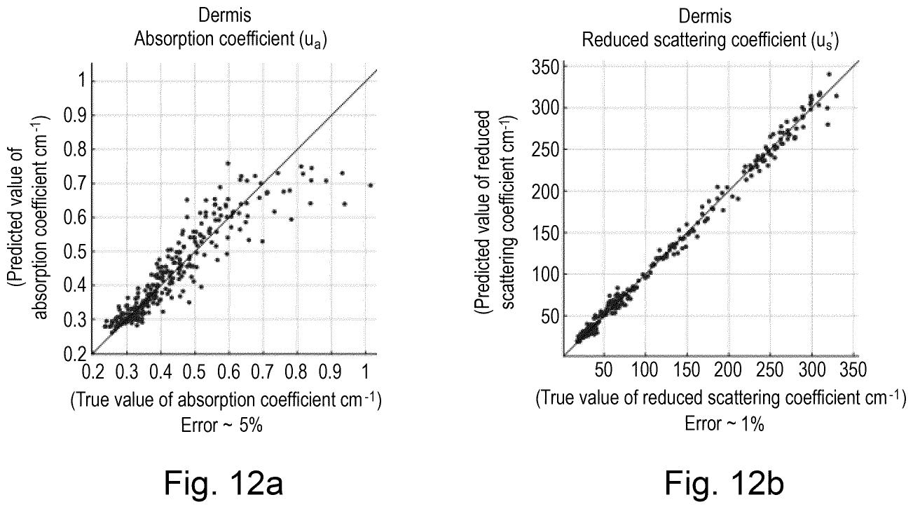

FIGS. 11a, 11b, 12a, 12b respectively show similar graphs but now the target is a two-layer model, each layer having variable thicknesses. For example, the example targets on which data is collected are targets having a varied thickness dermis and varied thickness epidermis (for example skin tissue from 25 different patients). Each layer of each different target has its own absorption coefficient and its own reduced scattering coefficient. As before, the data from which the graphs are generated is collected at sensor offsets of 0 μm, 250 μm, 500 μm, 750 μm, 1 mm, 1.25 mm, 1.5 mm, 1.5 mm, 1.75 mm, 2 mm, 2.25 mm, 2.5 mm. A laser with Gaussian distribution illuminates the target and the number of photons collected at the sensors at the different offsets is stored. These measurements are performed for varying values of absorption (uₐ) and reduced scattering coefficient (uₛ′) (both in cm⁻¹) to collect a training data set. Once training dataset of spatial offset location with number of photons arriving at the collector is sufficiently large, the model is trained to predict absorption and reduced scattering coefficient using the training data set, i.e. using only the photon count dataset. In the example graphs of FIGS. 11a-12b, a 5-fold cross validation approach is used. In addition, as the variable layer thickness results in increased errors, the error rate may be reduced by illuminating the target with a variety of different wavelengths, for example 11 different wavelengths between 650-850 nm. The additional variable provided by the different wavelengths reduces the coefficient prediction error rates to 11%, 8%, 5% and 1% respectively for the epidermis and dermis layers.

In each of the above cases, it is assumed that scattering is elastic.

Thus, the inventors have determined that the optical module of the present disclosure may be used to obtain photon counts that in turn may be used to predict absorption and reduced scattering coefficients. In turn, these may be used to estimate physical/optical parameters of the target samples and perform turbidity corrections of measured Raman spectra in the manner described above. Thus a miniaturized optical module is provided that is able to perform SORS on a target to obtain results that are not substantially affected by heterogeneity in the target.

Although the present disclosure has been described in terms of preferred embodiments as set forth above, it should be understood that these embodiments are illustrative only and that the claims are not limited to those embodiments. Those skilled in the art will be able to make modifications and alternatives in view of the disclosure that are contemplated as falling within the scope of the appended claims. Each feature disclosed or illustrated in the present specification may be incorporated in the invention, whether alone or in any appropriate combination with any other feature disclosed or illustrated herein.

For example, whilst the term mounted on has been used herein in connection with the sensors and laser sources being mounted on a substrate, it is envisaged that this includes incorporated and/or integrated the sensors and/or laser sources with the substrate and/or ASIC if present as part of a die manufacturing process.

For example, the offset dimensions will be application dependent but it is envisaged that offsets of around 0.1-3 mm may be used. Thus, once the dimensions of the integrated circuit and substrate are taken into account, a SORS enabled chip of around 5×5 mm² is envisaged. These dimensions provide an optical module which is suitably miniaturized for incorporation into wearable devices. It will be appreciated that other dimensions are also envisaged.

For example, the term substantially opaque means that a filter has an optical density around the range of 6-12 to stop wavelengths outside of its permitted band.

The present disclosure also envisages that, depending on the Raman excitation energies of the target molecules, multiple laser sources may be used with all the above embodiments to excite the target at multiple wavelength bands. Thus whilst multiple laser sources having emission wavelengths of at or between the narrowband values of 600 nm and 785 nm are described above, other wavelengths and wavelength bands are also envisaged. For example, each laser source of the plurality of laser sources may have a different wavelength.

The present disclosure also envisages that, in order to provide a fully miniaturized and integrated solution, the laser sources of all embodiments may comprise a laser diode, edge emitter, or vertical cavity surface emitting lasers (VCSEL) integrated with the substrate during a die manufacturing process. Similarly, the one or more sensors of the plurality of sensors may comprise a photo diode, a single photon avalanche diode (SPAD), an avalanche photo diode, a silicon photomultiplier (SiPM), a charge coupled device (CCD), or a MEMS photomultiplier integrated with the substrate, and/or ASIC if present. Advantageously, these components may easily be integrated with or in the substrate to further enhance the ease at which the device of the present disclosure may be mass-produced in semiconductor device fabrication facilities in high volumes compared to known Raman spectrometers which often require manual assembly which is slower and more expensive.

The term integrated circuit as used herein may refer to a set of electronic circuits integrated on semiconductor substrate thereby forming a microchip wherein all the circuit elements are inseparably associated and electrically interconnected so that the integrated circuit is considered to be indivisible as will be appreciated by the skilled person. The integrated circuit may in some implementations comprise a general purpose processor which is in contrast to the implementations provided with an application specific integrated circuit which refers to an integrated circuit customized for the particular use specified herein. The integrated circuit may be provided with a memory or storage comprising a computer program which when executed by the set of electronic circuits and/or processor causes the integrated circuit to perform the steps described herein.

The present disclosure also envisages that, for all embodiments, the first and second filters may comprise multiple layers of filters, each filter may comprise two filters layered on top of each other, to ensure such noise and signals from other molecules which are not molecules are interest are strongly attenuated and do not reach the plurality of sensors. For example, the first and second filters may comprise one or more dichroic filters and/or have an optical density value of between 10-12 for wavelengths outside the wavelength bands of interest.

The present disclosure also envisages that, for all embodiments, support structures may be used to position and enclose the components of the optical module on the substrate. Such support structures may be formed with, for example, injection molding and/or 3D printing and/or may be machined to alter their dimensions during optical calibration of the device. Such support structures may be opaque to electromagnetic radiation to ensure environmental noise at the plurality of sensors is reduced.

The present disclosure also envisages that, for all embodiments, the optical module may have a volume of under 2 cm³, for example between 1-2 cm³ or even 20-100 mm³ made possible by omission of the diffraction grating of known Raman spectrometers, thus allowing the optical module to be incorporated into portable and/or wearable devices such as smart watches, smart phones, heart rate monitors, and other vital sign monitors in point-of-care environments and/or sport settings. Similarly, the optical module may be incorporated into mobile devices such as smart phones and/or into attachments for mobile devices. Thereby making such devices Raman spectroscopy capable for the above described and other use cases where relative concentrations of specific, known molecules are required.

In the context where the target is skin tissue, the optical module of the present disclosure may be used to implement one or more of the following steps as initiated and controlled by the integrated circuit to determine skin depth or dermis layer at which optimum signal to noise ratio occurs:
(i) measuring SORS curves at every sensor/diode distance from the laser source;
(ii) performing layer analysis to determine the optimal distance where signal to noise is highest and the distance the signal is deemed background noise;
(iii) performing weighted subtraction of the Raman intensities at one or more of the measured wavelengths (corresponding to the filter spectral ranges);
(iv) performing multivariate curve resolution or band-target entropy minimization and/or apply other turbidity correction models;
(v) determine the target material type by analysing the Raman intensities at one or more of the measured wavelengths;
(vi) determining concentrations of one or more analytes at the determined signal to noise optimum sensor distance and effective optical skin depth.

### REFERENCE NUMERAL LIST

101 Raman spectra
102 Raman spectra
200 optical module
201 laser source
202 substrate
203 electromagnetic radiation
204 target
205a lens
205b lens
206 plurality of sensors
207 electromagnetic radiation
208 plurality of filters
209 support structures
210 ASIC
211 PCB
212a concentric ring
212b concentric ring
212c concentric ring

1. An optical module for spatially offset Raman spectroscopy, the optical module comprising:
a laser source mounted on a substrate and configured to emit electromagnetic radiation at a target;
a plurality of sensors mounted on the substrate and configured to detect electromagnetic radiation scattered from a plurality of depths in the target; and
a first plurality of filters, each disposed over one or more of the plurality of sensors,
wherein, the plurality of sensors and filters are arranged on the substrate at spatially offset positions from the laser source; and
wherein the first plurality of filters are substantially transparent to a first wavelength band corresponding to a Raman scattering wavelength of a first molecule of the target and substantially opaque to wavelengths outside the first wavelength band.

2. An optical module according to claim 1, wherein the substrate comprises an integrated circuit configured to:
determine a photon count at each of the plurality of sensors from the detected electromagnetic radiation at each of the plurality of sensors; and
use the determined photon counts to estimate an absorption coefficient and a reduced scattering coefficient of the target at each spatially offset position.

3. An optical module according to claim 2, wherein the integrated circuit is further configured to:
compare the estimated absorption coefficients and/or reduced scattering coefficients of the target with previously determined absorption coefficients and/or reduced scattering coefficients associated with one or more known samples to estimate physical information about the target.

4. An optical module according to claim 3, wherein said comparing by said integrated circuit comprises applying a trained model to said determined photon counts.

5. An optical module according to claim 2, wherein the integrated circuit is further configured to:
identify which of the plurality of sensors has a highest signal to noise ratio for the detected Raman scattering signals associated with said first molecule.

6. An optical module according to claim 2, wherein the integrated circuit is further configured to estimate a turbidity of the target and correct an output signal of one or more of said plurality of sensors to compensate for said estimated turbidity.

7. An optical module according to claim 2, wherein the integrated circuit is further configured to identify a modulating component in an output signal of one or more of said plurality of sensors associated with a periodic change in dimensions and/or composition of the target.

8. An optical module according to claim 1 wherein said laser source, said plurality of sensors, and said plurality of filters are integrated with said integrated circuit.

9. An optical module according to claim 2, wherein the integrated circuit comprises an ASIC.

10. An optical module according to claim 1, wherein the plurality of sensors are arranged in a line beginning at the laser source.

11. An optical module according to claim 1, wherein the plurality of sensors are arranged in a plurality of radial directions around the laser source in concentric, center symmetric patterns.

12. (canceled)

13. An optical module according to claim 1, wherein the plurality of sensors are arranged at unequal distances from each other in said line or in each said radial direction.

14. An optical module according to claim 1, wherein said plurality of sensors comprises at least three sensors in said line or in each said radial direction.

15. An optical module according to claim 1, comprising further pluralities of filters, each disposed over one or more of the plurality of sensors, wherein the further pluralities of filters are substantially transparent to a further wavelength bands corresponding to a Raman scattering wavelength of further molecules of the target and substantially opaque to wavelengths outside said further wavelength bands.

16. An optical module according to claim 15, wherein the integrated circuit is configured to:
estimate a ratio of signal strength between a detected Raman scattering signal of the first molecule and of one or more of said further molecules of the target at each of said spatially offset positions; and
compare the estimated ratios with previously determined ratios associated with one or more samples of known physical parameters to estimate one or more physical parameters of the target.

17. An optical module according to claim 16, wherein the physical parameter comprises a thickness of one or more layers of the target.

18. An optical module according to claim 15, wherein the laser source is configured to emit electromagnetic radiation in a plurality of wavelengths, and wherein the integrated circuit is further configured to:
estimate said absorption coefficient and a reduced scattering coefficient of the target from determined photon counts for each of said plurality of wavelengths.

19. An optical module according to claim 15, wherein the first plurality of filters are disposed over one or more of the plurality of sensors in a first radial direction from the laser source, and wherein the further plurality of filters are disposed over one or more of the plurality of sensors in further radial directions from the laser source.

20. (canceled)

21. An optical module according to claim 1, wherein said first and/or further plurality of filters comprise angular filters defining a field of view for each sensor of less than 28 degrees.

22. An optical module according to claim 2, wherein the integrated circuit is further configured to:
control the laser source to emit modulated electromagnetic radiation at the target and to demodulate the detected electromagnetic radiation scattered from the target.

23. (canceled)
